(19) United States
(12) Patent Application Publication
(10) Pub. No.: US 2014/0128725 A1
(43) Pub. Date: May 8, 2014

(54) NEONATE'S INCUBATOR AND MRI DOCKING-STATION

(71) Applicant: ASPECT IMAGING LTD., Shoham (IL)

(72) Inventor: Uri RAPOPORT, Moshav Ben Shemen (IL)

(73) Assignee: ASPECT IMAGING LTD., Shoham (IL)

(21) Appl. No.: 13/903,050

(22) Filed: May 28, 2013

Related U.S. Application Data
(60) Provisional application No. 61/723,806, filed on Nov. 8, 2012.

(51) Int. Cl. A61G 11/00; A61B 6/03; G01R 33/60; A61B 6/04; G01R 33/44; A61B 5/055; A61B 8/00

(52) U.S. Cl. CPC A61G 11/006; A61B 5/0555; A61B 6/032; A61B 8/40; A61B 6/0428; G01R 33/441; G01R 33/60
USPC 600/415; 600/407; 600/425; 600/437

(57) ABSTRACT

A docking-station useful for providing a neonate predefined, continuous, stabilized and non-interrupted life-support environmental conditions, comprising of: (i) a neonate incubator, having at least one first opening and a life support system, and (ii) an imaging-device, having a scanning chamber with at least one second opening. The docking-station is configured such that said neonate incubator first opening and said scanning chamber second opening are juxtapose-able so as to reversibly hermetically communicate; thereby providing said neonate predefined, continuous, stabilized, non-interrupted life-support environmental conditions during the entire process of scanning.

### FIELD OF THE INVENTION

The present invention generally pertains to a docking-station of (i) neonates' and premature-baby's incubator and (ii) scanning or imaging devices docking-station. More specifically, the present invention relates to an interfacing system and a reversible method thereof, for providing neonates and premature-babies a predefined, continuous, stabilized, non-interrupted environment within scanning or imaging-devices, such as MRI & CT, during the entire process of imaging or scanning, usually having an ambient environment unsuitable for said neonates and premature-baby.

### BACKGROUND OF THE INVENTION

As stated by Paly et al., (See Paley et al., An MR-compatible neonatal incubator, The British Journal of Radiology 85 (2012) 952-958), neonatal imaging is of growing importance as the effects of premature birth are becoming better controlled and babies are surviving from earlier gestational ages. Prematurity can often lead to consequences for the CNS and other parts of the body and MR is highly relevant for imaging this population as it is non-invasive and so allows follow up scanning There have been two main approaches to imaging neonates who require monitoring and preservation of temperature away from the care unit: (a) a dedicated MR system on the intensive care unit where care and support can be easily provided. (b) a neonatal incubator with full support equipment to transport the baby to a central hospital based MR system. A number of prototype incubators and a commercial system have previously been reported but all these systems have one or more disadvantages in terms of cost, complexity and patient visibility during imaging.

Moreover, a group of standards are applicable for neonates and premature-baby, and those standards consisting of IEC 6061-2-19 Standard Clauses 12 201.12.101 (Stability of incubator); IEC 201.12.102 (Uniformity of incubator temperature); IEC 201.12.103 (Accuracy of skin temperature sensor); IEC 201.12.1.104 (accuracy between skin temperature and control temperature during removal, transportation and insertion within said MRD device); IEC 201.12.1.108 (overshoot time) and/or IEC 12 201.12.1.107 (warm up time). The standards characterized standardize incubators as those adapted to (i) maintain a steady temperature condition within a range of about ±0.5 degrees Celsius; (ii) maintain steady temperature condition over a period of at least one hour when checked at about 32 degrees Celsius and about 36 degrees Celsius; (iii) maintain steady temperature condition during baby control such that the skin temperature of the neonate does not differ from said predetermined steady temperature condition by more than 0.7 degrees Celsius; (iv) maintain temperature of incubator at 12 degrees Celsius above ambient temperature, supply voltage being equal to the rated voltage, said incubator operating as an air controlled infant incubator, said infant incubator is switched on starting from cold condition, and time for the incubator temperature to rise by 11 degrees Celsius is measured, the warm up time of said capsule substantially conforms with warm up time of said incubator; (v) maintain an overshoot temperature of 2 degrees Celsius temperature within said incubator; (vi) maintain said temperature during a 15 minute steady state temperature condition restoration period; and (vii) maintain substantially similar temperature conditions to that of said incubator when said infant incubator is operated as an air controlled incubator at a control temperature of 32 degrees until said steady state temperature is reached, and the temperature is then adjusted to a control temperature of 36 degrees Celsius, the overshoot of incubator and the time to reach the new steady temperature condition from the first passage of 36 degrees Celsius being measured.

There is a long felt unmet need for means and methods of providing a predefined, continuous, stabilized, non-interrupted environment within scanning or imaging-devices, such as MRI & CT, during the entire process of imaging or scanning.

### SUMMARY OF THE PRESENT INVENTION

It is one object of the present invention to provide a docking-station 100, useful for providing a neonate 300 a predefined, continuous, stabilized and non-interrupted, life-support environmental conditions, comprising of:
a) a neonate incubator 110, having at least one first opening 170 and a life support system 180 and
b) an imaging-device 210, having a scanning chamber 220 with at least one second opening 230;

wherein said docking-station 100 is configured such that said neonate incubator first opening 170 and said scanning chamber 220 second opening 230 are juxtapose-able so as to reversibly hermetically communicate, thereby providing said neonate 300 said predefined, continuous, stabilized, non-interrupted life-support environmental conditions, during the entire process of scanning.

It is another object of the present invention to provide the docking-station 100 as defined above, wherein said neonate incubator 110 comprises:
a) a movable bed 130, configured for accommodating said neonate 300;
b) mechanical-means 140 for conveying said movable bed 130 accommodating said neonate 300 from said incubator 110 into said scanning chamber 220 of said imaging-device 210, and conveying said movable bed 130 accommodating said neonate 300 out of said scanning chamber 220 back into said neonate incubator 110;
c) a front door 150, for opening and closing said neonate incubator first opening 170, configured to allow said translating of said movable bed 130 accommodating said neonate 300; and
d) sealing-means 160 surrounding said neonate incubator first opening 170 of said neonate incubator 110, configured to be reversibly and hermetically connected around said second opening 230 of said scanning chamber 220 of said imaging-device 210 and adapted to hermetically seal said scanning chamber 220 of said imaging-device 210, while said incubator door 150 is closed; and seal said scanning chamber 220 to said connected neonate incubator 110, while said neonate incubator door 150 is open.

It is another object of the present invention to provide the docking-station 100 as defined above, wherein said scanning chamber 220 of said imaging-device 210 comprises a sealable aperture 240 configured for adjusting said environment of said scanning chamber 220.

It is another object of the present invention to provide the docking-station 100 as defined above, wherein said imaging-device 210 is a scanning device selected from the group consisting of: MRI, NMR, ESR, NQR, CT, US and M2™.

It is another object of the present invention to provide the docking-station 100 as defined above, wherein said closed circuit life support system 180 of said neonate incubator 110 is mounted or mountable on said neonate incubator 110.

It is another object of the present invention to provide the docking-station 100 as defined above, wherein said closed circuit life support system of said incubator 110 is replaced by a central life support system.

It is another object of the present invention to provide the docking-station 100 as defined above, wherein said environment characteristics of said scanning chamber 220 of said imaging-device 210 are supplied by said neonate incubator's mounted closed circuit life support system 180.

It is another object of the present invention to provide the docking-station 100 as defined above, wherein said environment characteristics of said scanning chamber 220 of said imaging-device 210 are supplied by a central life support system.

It is another object of the present invention to provide the docking-station 100 as defined above, wherein the characteristics of said Life Support Environment are selected from the group consisting at least one of: oxygen, temperature, humidity, gas and fluid exchange, drug delivery, perfusions, waste removal transfusions, anesthetic gas and any combination thereof.

It is another object of the present invention to provide the docking-station 100 as defined above, wherein said sealing-means 160 is a gasket or a plurality of gaskets.

It is another object of the present invention to provide the docking-station 100 as defined above, wherein said front door 150 is selected from a group consisting of: sliding door, axis door, concertina door and gas-tight flaps.

It is another object of the present invention to provide the docking-station 100 as defined above, wherein said neonate 300 is fastened to said movable bed 130 by fasteners made of any material invisible to magnetic fields.

It is another object of the present invention to provide the docking-station 100 as defined above, wherein said neonate incubator 110 is mounted on a movable cart 190.

It is another object of the present invention to provide the docking-station 100 as defined above, wherein said neonate incubator 110 and its life support system 180 are mounted or mountable on a movable cart 190.

It is another object of the present invention to provide the docking-station 100 as defined above, wherein said imaging device 210 and said neonate incubator 110 are connectable by a communicating passage selected from a group consisting of: a manifold, flexible passage, connecting sleeve, flexible sleeve, concertina like sleeve and any combination thereof.

It is another object of the present invention to provide a method for providing a neonate 300 a predefined, continuous, stabilized, non-interrupted life support environmental conditions, within an imaging-device 210, during the entire process of scanning; said method comprising the steps of:
a) obtaining: (i) a neonate incubator 110, having: at least one first opening 170 configured with a door 150, a movable bed 130, and (ii) an imaging-device 210, having a scanning chamber 220 with at least one second opening 230;
b) hermetically connecting said at least one first opening 170 of said neonate containing incubator 110 to said second opening 230 of ID 210 via said neonate containing incubator closed door 150;
c) adjusting the environmental characteristics of said ID's 210 scanning chamber 220 to that of said neonate containing incubator 110;
d) opening said door 150 of said neonate containing incubator 110;
e) translating said neonate's movable bed 130 into said ID's 210 scanning chamber 220; and
f) scanning said neonate 300;
   thereby providing said neonate 300 a predefined, continuous, stabilized, non-interrupted life support environment, within an imaging-device 210, during the entire process of scanning.

It is another object of the present invention to provide the method as defined above, further comprising steps of:
a) opening said front door 150 of said incubator 110;
b) translating said neonate's movable bed 130 out of said ID's 210 scanning chamber 220 and back to said incubator 110; and
c) closing said front door 150 of said incubator 110.

It is another object of the present invention to provide the method as defined above, further comprising step of detaching said incubator 110 from said imaging-device 210.

It is another object of the present invention to provide the method as defined above, wherein said incubator 110 further comprising:
a) a life support system 180;
b) mechanical-means 140 for conveying said movable bed 130 accommodating said neonate 300 from said incubator 110 into said scanning chamber 220 of said imaging-device 210, and conveying said movable bed 130 accommodating said neonate 300 out of said scanning chamber 220 back into said incubator 110; and
c) sealing-means 160 surrounding said incubator first opening 170 of said incubator 110, configured to be reversibly and hermetically connected around the second opening 230 of said scanning chamber 220 of said imaging-device 210 and adapted to seal said scanning chamber 220 of said imaging-device 210, while said incubator door 150 is closed and seal said scanning chamber 220 to said connected incubator 110, while said incubator door 150 is open.

It still an object of the present invention to provide the method as defined above, wherein said scanning chamber 220 of said imaging-device 210 further comprising a sealable aperture 240 configured for adjusting said environment of said scanning chamber 220.

Lastly an object of the present invention to provide the method as defined above, further comprising step of equalizing the environment of said sealed scanning chamber 220 of said imaging-device 210, via said aperture 240 in said scanning chamber 220, to ambience with the predetermined life support environment of said incubator 110, while said incubator's front door 150 remains closed.

### BRIEF DESCRIPTION OF THE DRAWINGS

These and further objects of the present invention will become more apparent upon consideration of the accompanying drawings wherein:

FIG. 1 illustrates parts of the docking station 100;

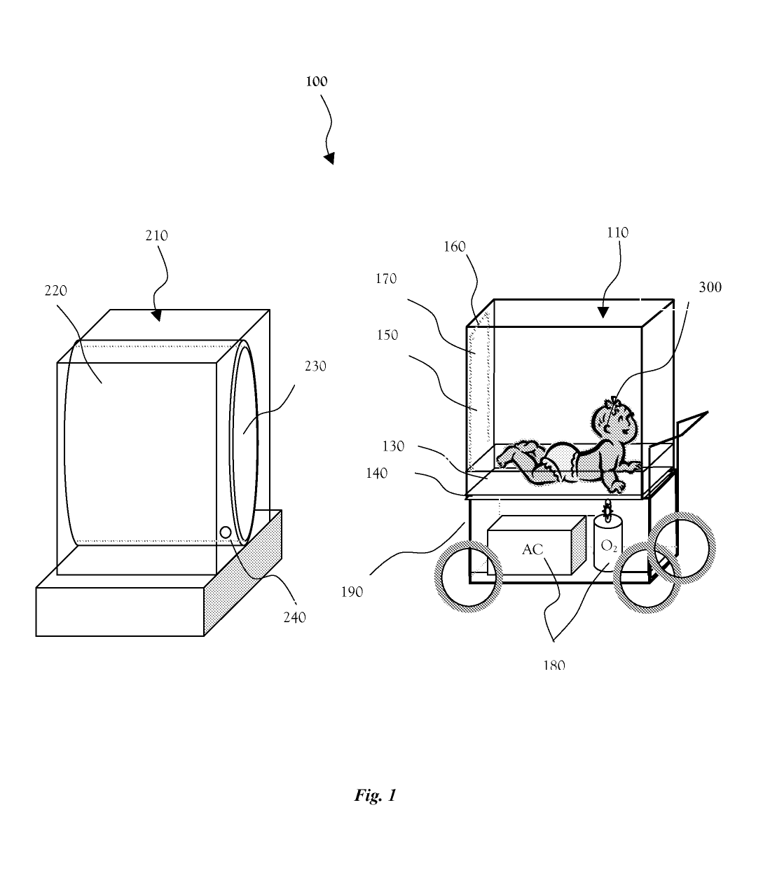

FIG. 2 illustrates connection of the incubator 110 to the imaging device 210;

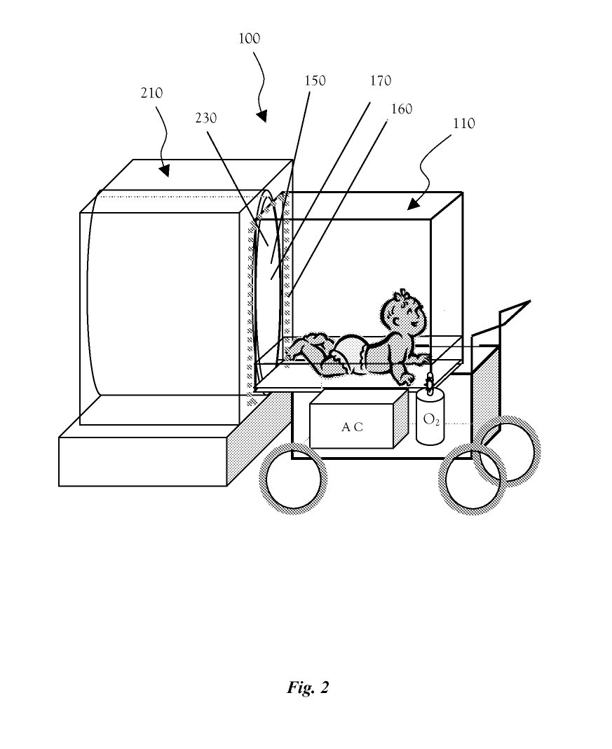

FIG. 3 illustrates translation of the movable bed 130 into the imaging device scanning chamber 220;

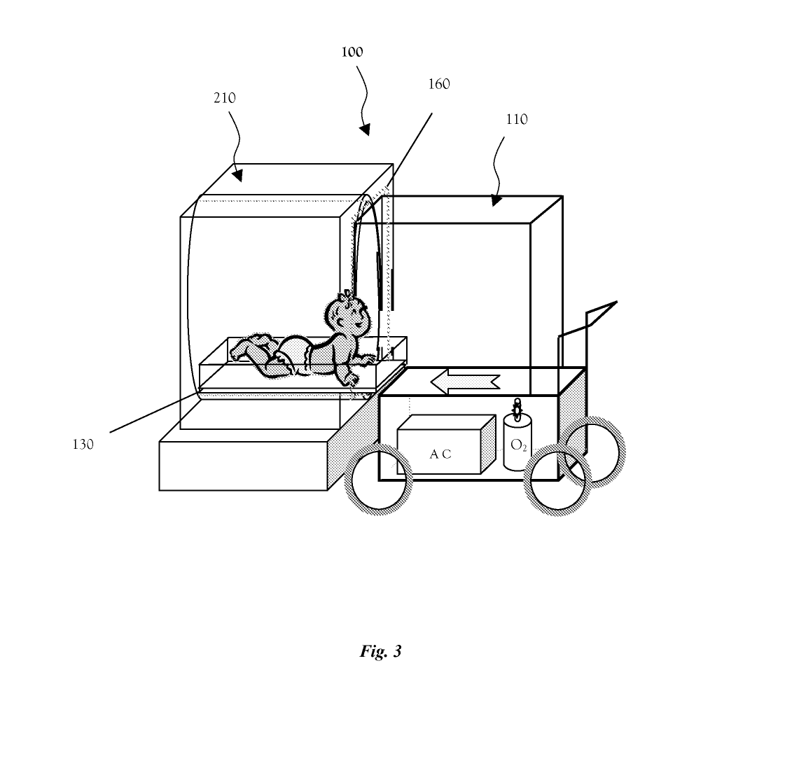

FIG. 4 illustrates translation of the movable bed 130 out of the imaging device scanning chamber 220;

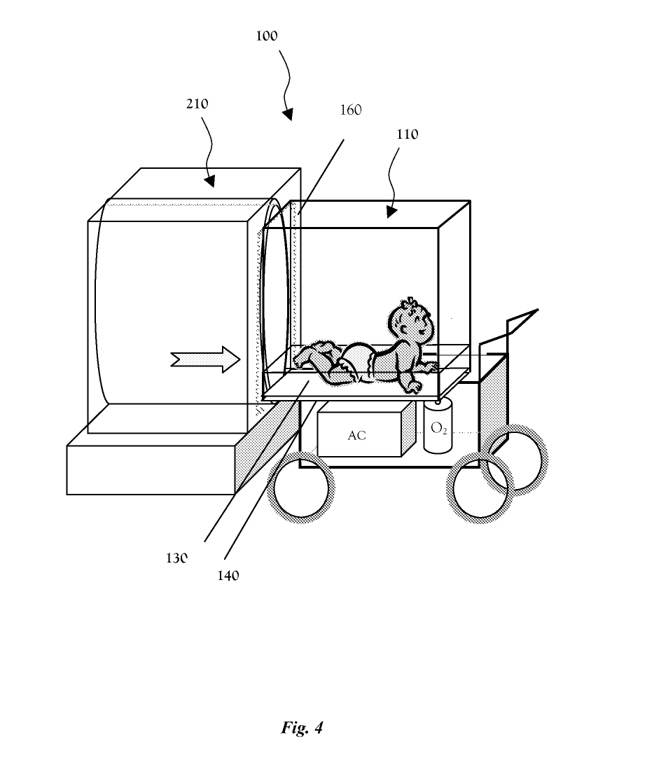

FIG. 5 illustrates detachment of the incubator 110 from the imaging device 210;

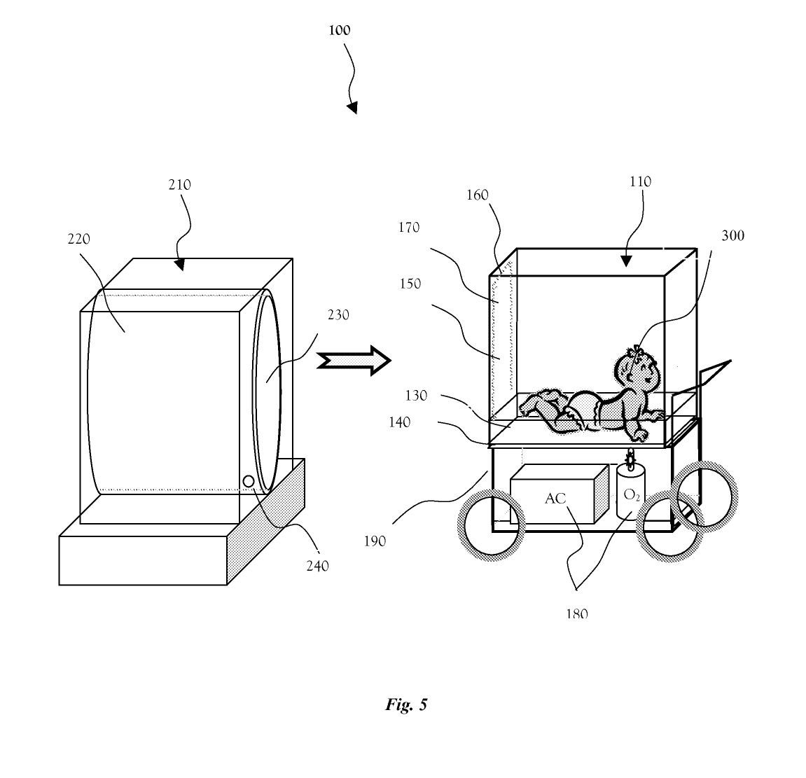

FIG. 6 illustrates the modules of the docking station 100;

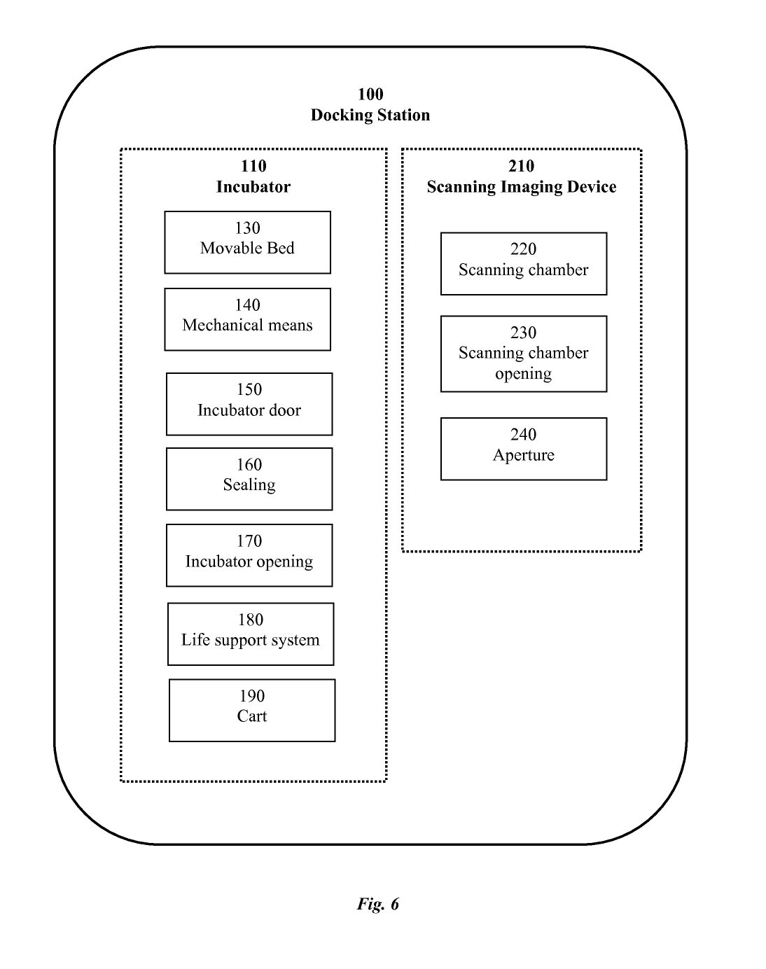

FIG. 7 illustrates the method steps.

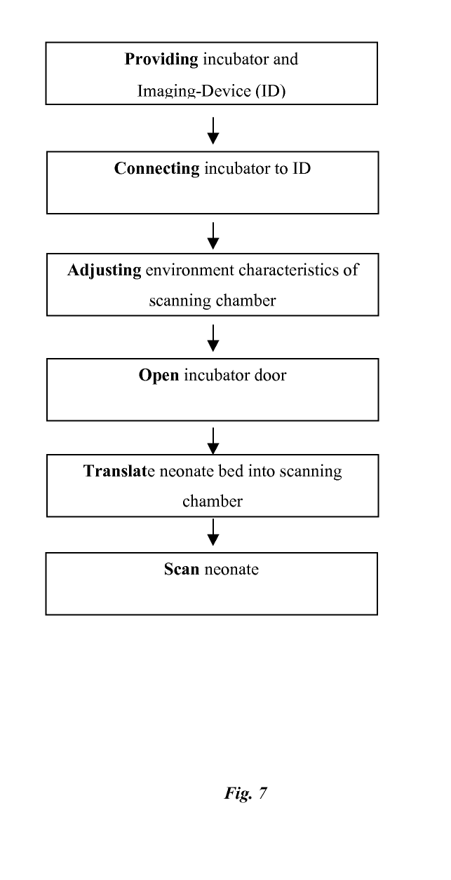

### DETAILED DESCRIPTION OF THE INVENTION

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a neonate's incubator and MEI docking system.

The term ‘magnetic resonance device’ (MRD) specifically applies hereinafter to any Magnetic Resonance Imaging (MRI) device, any Nuclear Magnetic Resonance (NMR) spectroscope, any Electron Spin Resonance (ESR) spectroscope, any Nuclear Quadruple Resonance (NQR) or any combination thereof. The term, in this invention, also applies to any other analyzing and imaging instruments comprising a volume of interest, such as computerized tomography (CT), ultrasound (US) etc. The MRD hereby disclosed is optionally a portable MRI device, such as the ASPECT-MR Ltd commercially available devices, or a commercially available non-portable device. Moreover, the term ‘MRD’ interchangeably refers in general to any non-incubator medical devices, at least temporary accommodating the neonate.

As used herein, the term “neonate” generally refers to any object or living creature, such as human being or other mammal and preferably refers to babies or premature babies.

As used herein, the term “incubator” may include isolate-like devices which are self-contained incubator units that provides a controlled heat, humidity, and oxygen microenvironment for the isolation and care of premature and low-birth weight neonates. The apparatus is often made of a clear plastic material and has a large door and portholes for easy access to the infant with a minimum of heat and oxygen loss. A servo control mechanism constantly monitors the infant's temperature and controls the heat within the unit

As used herein, the term “bed” refers to any object, upon a surface of which the neonate rests before and during acquisition of the magnetic resonance images by an MRI system. As a non-limiting example, the surface on which the neonate rests is the upper surface of the object and is essentially planar. The bed may be translatable to a position located external to the MRI

As used herein, the terms “ambience with” means that the Life Support Environment is essentially similar in the selected Life Support Environmental parameter between the neonate incubator and the scanning chamber of the imaging device. The life support environment in provided by e.g. an open system or closed system.

The term ‘about’ refers herein to a value of ±25% of the defined measure.

Reference is now made to a docking-station 100 comprising of: A neonate incubator 110 having at least one first opening 170 and a closed circuit life support system 180 and an imaging-device 210 having a scanning chamber 220; The docking station 100 is adapted such that said incubator and said scanning chamber 220 are adapted to reversibly hermetically communicate to provide said neonate 300 predefined, continuous, stabilized, non-interrupted environmental conditions during the entire process of scanning.

Reference is now made to the aforementioned docking-station 100 having an incubator 110 comprising; a movable bed 130, configured for accommodating said neonate 300; mechanical-means 140 for conveying said movable bed 130 accommodating said neonate 300 from said incubator 110 into said scanning chamber 220 of said imaging-device 210, and conveying said movable bed 130 accommodating said neonate 300 out of said scanning chamber 220 back into said incubator 110. Furthermore, the aforementioned incubator has a front door 150, for opening and closing the incubator first opening 170, and the front door is configured to allow translating of said movable bed 130 accommodating said neonate 300. There is a sealing-means 160 surrounding the incubator first opening 170 of the incubator 110, which is configured to be reversibly and hermetically connected around the second opening 230 of the scanning chamber 220 of the imaging-device 210 and is adapted to hermetically seal the scanning chamber 220 of the imaging-device 210 while the incubator door 150 is closed and also seal the scanning chamber 220 to the now connected incubator 110, while the incubator door 150 is open.

Reference is now made to the aforementioned docking-station 100 which includes a scanning chamber 220 of the imaging-device 210 comprising a sealable aperture 240 configured for adjusting the environment of the scanning chamber 220.

Reference is now made to the aforementioned docking-station 100 having an imaging-device 210 which is a scanning device selected from the group consisting of: MRI, NMR, ESR, NQR, CT, US and M2™.

Reference is now made to the aforementioned docking-station 100 having a closed circuit life support system 180 of the incubator 110 mounted on the incubator 110.

Reference is now made to the aforementioned docking-station 100 having the closed circuit life support system of the incubator 110 replaced by a central life support system.

Reference is now made to the aforementioned docking-station 100 having the environmental characteristics of the scanning chamber 220 of the imaging-device 210 supplied by the incubator's mounted closed circuit life support system 180.

Reference is now made to the aforementioned docking-station 100 having the environment characteristics of the scanning chamber 220 of the imaging-device 210 supplied by a central life support system.

Reference is now made to the aforementioned docking-station 100 characterized in that the Life Support Environment is selected from the group of parameters consisting of at least one of: oxygen, temperature, humidity, gas and fluid exchange, drug delivery, perfusions, waste removal transfusions, anesthetic gas and any combination thereof.

Reference is now made to the aforementioned docking-station 100 having sealing-means 160 comprising of a gasket or a plurality of gaskets.

Reference is now made to the aforementioned docking-station 100 having the front door 150 selected from a group consisting of: sliding door, axis door, concertina door and gas-tight flaps.

Reference is now made to the aforementioned docking-station 100 adapted such that the neonate 300 is fastenable to said movable bed 130 by fasteners made of any material invisible to magnetic fields.

Reference is now made to the aforementioned docking-station 100 having an incubator 110 mounted or mountable on a movable cart 190.

Reference is now made to the aforementioned docking-station 100 having the life support system 180 mounted or mountable on a movable cart 190.

Reference is now made to the aforementioned docking-station 100 having the incubator 110 and its life support system 180 mounted or mountable on a movable cart 190.

Reference is now made to the aforementioned docking-station 100 having an imaging device 210 and said incubator 110 connected or connectable by a communicating passage selected from a group consisting of: a manifold, flexible passage, connecting sleeve, flexible sleeve, concertina like sleeve and any combination thereof.

Reference is now made to a method for providing a neonate 300 a predefined, continuous, stabilized, non-interrupted life support environment, within an imaging-device 210, during the entire process of scanning The method comprises the steps of: obtaining a docking-station 100 comprising: a neonate incubator 110 having at least one first opening 170 and a closed circuit life support system 180 and an imaging-device 210 having a scanning chamber 220; The aforementioned incubator 110 comprises: a movable bed 130, configured for accommodating the neonate 300; mechanical-means 140 for conveying the movable bed 130 accommodating said neonate 300 from the incubator 110 into the scanning chamber 220 of the imaging-device 210, and conveying the movable bed 130 accommodating the neonate 300 out of the scanning chamber 220 back into the incubator 110.

The incubator further comprises a front door 150, for opening and closing the incubator first opening 170, configured to allow translation of the movable bed 130 accommodating said neonate 300; and sealing-means 160 surrounding the incubator first opening 170. The sealing means is configured to be reversibly and hermetically connected around the second opening 230 of the scanning chamber 220 of the imaging-device 210 and it is adapted to seal the scanning chamber 220 of the imaging-device 210, while said incubator door 150 is closed and seal the scanning chamber 220 to the connected incubator 110, while the incubator door 150 is open.

Having obtained the aforementioned, the following steps of the method comprise; connecting the incubator's front door 150 to the chamber second opening 230 of the imaging-device 210, while the incubator's front door 150 remains closed; hermetically sealing the scanning chamber 220 of the imaging-device 210, by sealing means 160; opening the aperture 240 of the scanning chamber 220 of the imaging-device 210; equalizing the environment of said sealed scanning chamber 220 of the imaging-device 210, via the aperture 240 in said scanning chamber 220, to ambience with the predetermined Life Support Environment (LSE) of the incubator 110, while the incubator's front door 150 remains closed; opening the front door 150 of the incubator 110; translating the movable bed 130 accommodating the neonate 300 out of the incubator 110 and into the scanning chamber 220 of the imaging-device 210; scanning the neonate 300 by the imaging-device 210; translating the movable bed 130 accommodating the neonate 300 out of the scanning chamber 220 of the imaging-device 210 and back to said incubator 110.

Further steps include closing said front door 150 of the incubator 110; closing the aperture 240 of the scanning chamber 220 of the imaging-device 210; detaching the incubator 110 from the imaging-device 210 thereby providing predefined, continuous, stabilized, non-interrupted life support environment, within an imaging-device 210, during the entire process of scanning.

1. A docking-station 100 useful for providing a neonate 300 a predefined, continuous, stabilized and non-interrupted, life-support environmental conditions, comprising of:
a) a neonate incubator 110, having at least one first opening 170 and a life support system 180 and
b) an imaging-device 210, having a scanning chamber 220 with at least one second opening 230;
wherein said docking-station 100 is configured such that said neonate incubator first opening 170 and said scanning chamber 220 second opening 230 are juxtapose-able so as to reversibly hermetically communicate, thereby providing said neonate 300 said predefined, continuous, stabilized, non-interrupted life-support environmental conditions, during the entire process of scanning.

2. The docking-station 100 according to claim 1, wherein said neonate incubator 110 comprises:
a) a movable bed 130, configured for accommodating said neonate 300;
b) mechanical-means 140 for conveying said movable bed 130 accommodating said neonate 300 from said incubator 110 into said scanning chamber 220 of said imaging-device 210, and conveying said movable bed 130 accommodating said neonate 300 out of said scanning chamber 220 back into said neonate incubator 110;
c) a front door 150, for opening and closing said neonate incubator first opening 170, configured to allow said translating of said movable bed 130 accommodating said neonate 300; and
d) sealing-means 160 surrounding said neonate incubator first opening 170 of said neonate incubator 110, configured to be reversibly and hermetically connected around said second opening 230 of said scanning chamber 220 of said imaging-device 210 and adapted to hermetically seal said scanning chamber 220 of said imaging-device 210, while said incubator door 150 is closed; and seal said scanning chamber 220 to said connected neonate incubator 110, while said neonate incubator door 150 is open.

3. The docking-station 100 according to claim 1, wherein said scanning chamber 220 of said imaging-device 210 comprises a sealable aperture 240 configured for adjusting said environment of said scanning chamber 220.

4. The docking-station 100 according to claim 1, wherein said imaging-device 210 is a scanning device selected from the group consisting of: MRI, NMR, ESR, NQR, CT, US and M2™.

5. The docking-station 100 according to claim 1, wherein said closed circuit life support system 180 of said neonate incubator 110 is mounted or mountable on said neonate incubator 110.

6. The docking-station 100 according to claim 1, wherein said closed circuit life support system of said incubator 110 is replaced by a central life support system.

7. The docking-station 100 according to claim 3, wherein said environment characteristics of said scanning chamber 220 of said imaging-device 210 are supplied by said neonate incubator's mounted closed circuit life support system 180.

8. The docking-station 100 according to claim 3, wherein said environment characteristics of said scanning chamber 220 of said imaging-device 210 are supplied by a central life support system.

9. The docking-station 100 according to claim 1, wherein the characteristics of said Life Support Environment are selected from the group consisting at least one of: oxygen, temperature, humidity, gas and fluid exchange, drug delivery, perfusions, waste removal transfusions, anesthetic gas and any combination thereof.

10. The docking-station 100 according to claim 2, wherein said sealing-means 160 is a gasket or a plurality of gaskets.

11. The docking-station 100 according to claim 2, wherein said front door 150 is selected from a group consisting of: sliding door, axis door, concertina door and gas-tight flaps.

12. The docking-station 100 according to claim 2, wherein said neonate 300 is fastened to said movable bed 130 by fasteners made of any material invisible to magnetic fields.

13. The docking-station 100 according to claim 2, wherein said neonate incubator 110 is mounted on a movable cart 190.

14. The docking-station 100 according to claim 5, wherein said neonate incubator 110 and its life support system 180 are mounted or mountable on a movable cart 190.

15. The docking-station 100 according to claim 2, wherein said imaging device 210 and said neonate incubator 110 are connectable by a communicating passage selected from a group consisting of: a manifold, flexible passage, connecting sleeve, flexible sleeve, concertina like sleeve and any combination thereof.

16. A method for providing a neonate 300 a predefined, continuous, stabilized, non-interrupted life support environmental conditions, within an imaging-device 210, during the entire process of scanning; said method comprising the steps of:
a) obtaining: (i) a neonate incubator 110, having: at least one first opening 170 configured with a door 150, a movable bed 130, and (ii) an imaging-device 210, having a scanning chamber 220 with at least one second opening 230;
b) hermetically connecting said at least one first opening 170 of said neonate containing incubator 110 to said second opening 230 of ID 210 via said neonate containing incubator closed door 150;
c) adjusting the environmental characteristics of said ID's 210 scanning chamber 220 to that of said neonate containing incubator 110;
d) opening said door 150 of said neonate containing incubator 110;
e) translating said neonate's movable bed 130 into said ID's 210 scanning chamber 220; and
f) scanning said neonate 300;
thereby providing said neonate 300 a predefined, continuous, stabilized, non-interrupted life support environment, within an imaging-device 210, during the entire process of scanning.

17. The method according to claim 16, further comprising steps of:
a) opening said front door 150 of said incubator 110;
b) translating said neonate's movable bed 130 out of said ID's 210 scanning chamber 220 and back to said incubator 110; and
c) closing said front door 150 of said incubator 110.

18. The method according to claim 17, further comprising step of detaching said incubator 110 from said imaging-device 210.

19. The method according to claim 16, wherein said incubator 110 further comprising:
a) a life support system 180;
b) mechanical-means 140 for conveying said movable bed 130 accommodating said neonate 300 from said incubator 110 into said scanning chamber 220 of said imaging-device 210, and conveying said movable bed 130 accommodating said neonate 300 out of said scanning chamber 220 back into said incubator 110; and
c) sealing-means 160 surrounding said incubator first opening 170 of said incubator 110, configured to be reversibly and hermetically connected around the second opening 230 of said scanning chamber 220 of said imaging-device 210 and adapted to seal said scanning chamber 220 of said imaging-device 210, while said incubator door 150 is closed and seal said scanning chamber 220 to said connected incubator 110, while said incubator door 150 is open.

20. The method according to claim 16, wherein said scanning chamber 220 of said imaging-device 210 further comprising a sealable aperture 240 configured for adjusting said environment of said scanning chamber 220.

21. The method according to claim 20, further comprising step of equalizing the environment of said sealed scanning chamber 220 of said imaging-device 210, via said aperture 240 in said scanning chamber 220, to ambience with the predetermined life support environment of said incubator 110, while said incubator's front door 150 remains closed.

22. The docking-station 100 according to claim 5, wherein said environment characteristics of said scanning chamber 220 of said imaging-device 210 are supplied by said neonate incubator's mounted closed circuit life support system 180.
